(19) United States
(12) Patent Application Publication
(10) Pub. No.: US 2020/0237331 A1
(43) Pub. Date: Jul. 30, 2020

(54) IMPROVEMENTS IN THE RADIOLOGICAL DETECTION OF CHRONIC THROMBOEMBOLIC PULMONARY HYPERTENSION

(71) Applicant: Bayer Aktiengesellschaft, Leverkusen (DE)

(72) Inventors: Karym EL SAYED, Berlin (DE); Simon Alexander TEAL, Berlin (DE); Eva-Maria QUAST, Potsdam (DE); Soeren HOERNIG, Berlin (DE); Mitesh DAFTARDAR, Berlin (DE); Matthias MÜHLENDYCK, Berlin (DE); Hans-Peter PODHAISKY, Berlin (DE); Barbara HINZMANN, Berlin (DE); Oliver SIEGLER, Berlin (DE); Levent KILIC, Berlin (DE)

(73) Assignee: Bayer Aktiengesellschaft, Leverkusen (DE)

(21) Appl. No.: 16/609,138

(22) PCT Filed: Apr. 26, 2018
(86) PCT No.: PCT/EP2018/060732

§ 371 Date: Oct. 28, 2019

(30) Foreign Application Priority Data
May 2, 2017 (EP) 17169079.5

(51) Int. Cl. A61B 6/00; A61B 6/03; G06T 7/00; G06T 11/00; G16H 50/20; G16H 30/20

(52) U.S. Cl. CPC A61B 6/5217; A61B 6/032; A61B 6/503; A61B 6/504; A61B 6/507; G06T 2207/30101; G06T 11/008; G16H 50/20; G16H 30/20; G06T 2207/10081; G06T 2207/30048; G06T 7/0012

(57) ABSTRACT

The present invention is concerned with the radiological identification of chronic thromboembolic pulmonary hypertension (CTEPH). The invention relates to a method, a computer system and a computer program product for automatically identifying signs of the presence of CTEPH in a person.

### CROSS REFERENCE TO RELATED APPLICATIONS

This application is a national stage application under 35 U.S.C. § 371 of International Application No. PCT/EP2018/060732, filed internationally on Apr. 26, 2018, which claims the benefit of European Application No. 17169079.5, filed May 2, 2017.

### FIELD OF THE INVENTION

The present invention relates to the radiological identification of chronic thromboembolic pulmonary hypertension (CTEPH). More specifically, it relates to a method, a computer system and a computer program product for automatically identifying signs of the presence of CTEPH in a person.

### BACKGROUND OF THE INVENTION

Chronic thromboembolic pulmonary hypertension (CTEPH) is a special form of pulmonary hypertension (PH). It is characterized by the infiltration of thrombi into the pulmonary arteries. They clog and constrict the vessels in a complete or partial manner and can be converted into connective tissue. In rare cases, there is the development of pulmonary hypertension with a poor prognosis.

The symptoms of CTEPH are non-specific. In the early stage, dyspnoea and fatigue may occur. The duration of the initial symptoms for diagnosis is on average 14 months, and during the course of this some patients are then already in an advanced stage of the disease. This underlines the need for an exact and timely diagnosis.

A timely diagnosis of CTEPH is also important because there are meanwhile options for the therapy of different forms of manifestation of this disease.

The preferred CTEPH therapy is operative pulmonary endarterectomy (PEA), by means of which it is possible to heal up to 70% of patients. Meanwhile, perioperative mortality at experienced centres is 2-4%. However, about 30-50% of all CTEPH patients are classified as non-operable. For these patients and for patients with persistent pulmonary hypertension after PEA, a medicament-based therapy was authorized for the first time at the beginning of 2014 in the form of Riociguat.

The gold standard for diagnosing or ruling out a case of CTEPH is ventilation/perfusion scintigraphy. The negative predictive value of perfusion scintigraphy is almost 100%; this means that a proper perfusion distribution rules out a case of CTEPH with a probability bordering on certainty.

However, the problem is that CTEPH is comparatively rare. As a result of the rarity and the complex diagnosis and differential diagnosis, CTEPH is underdiagnosed.

There is therefore a need for an early and uncomplicated identification of signs of the presence of CTEPH.

### SUMMARY OF THE INVENTION

According to some embodiments of the invention, a method is provided for identifying signs of the presence of CTEPH in a person, comprising:
receiving or retrieving one or more computed tomography images of the thorax of the person,
analysing the one or more computed tomography images by means of an image recognition software,
determining features in the one or more computed tomography images, which features indicate the presence of CTEPH,
calculating a probability of the presence of CTEPH on the basis of the features ascertained, and
communicating a message to the person and/or another person for further assessment of the case if the probability is above a defined threshold,
   wherein the steps mentioned run automatically as background processes in one or more computer systems.

According to some embodiments of the invention, a computer system is provided for identifying signs of the presence of CTEPH in a person, comprising:
means for the automatic receipt or retrieval of one or more computed tomography images of the thorax of the person,
means for the automated analysis of the one or more computed tomography images,
means for the automated identification of features in the one or more computed tomography images, which features indicate the presence of CTEPH,
means for automatically calculating a probability of the presence of CTEPH on the basis of the features ascertained, and
means for the automated communication of a message to the person and/or another person for further clarification of the finding.

According to some embodiments of the invention, a computer program product is provided, the computer program product comprising a data carrier on which there is stored a computer program which can be loaded into the memory of a computer system, wherein the computer program causes the computer system to execute the following steps:
receiving or retrieving one or more computed tomography images of the thorax of the person,
analysing the one or more computed tomography images by means of an image recognition software,
determining features in the one or more computed tomography images, which features indicate the presence of CTEPH,
calculating a probability of the presence of CTEPH on the basis of the features ascertained, and
communicating a message to the person and/or another person for further assessment of the case if the probability is above a defined threshold,
   wherein the steps run automatically as background processes on the computer system.

The method, computer system, and the computer program product according to some embodiments of the invention, are explained in more detail below. This explanation does not make a distinction between the individual subjects of the invention (method, computer system, computer program product). Instead, the following descriptions apply analogously to all subjects of the invention, irrespective of their context.

In the identification of signs of the presence of CTEPH in a person, some embodiments of the present invention focuses on the automated image analysis of computed tomography images of the thorax of the person.

Computed tomography (CT) is an X-ray method which depicts the human body in cross-sectional images (sectional imaging method). Compared to a conventional X-ray image, on which usually only coarse structures and bones are identifiable, CT images also capture in detail soft tissues with small differences in contrast. An X-ray tube generates a so-called X-ray fan beam, which penetrates the body and is attenuated to varying degrees within the body owing to the various structures, such as organs and bones. The receiving detectors opposite the X-ray emitter receive the signals of varying strength and forward them to a computer, which puts together cross-sectional images of the body from the received data. Computed tomography images (CT images) can be observed in 2D or else in 3D. For better differentiability of structures within the body of the person (e.g. vessels), a contrast agent can be injected into a vein before the generation of CT images.

Computed tomography is a commonly used method in the diagnosis of heart and lung diseases. Preferably, the CT images are multidetector CT images.

So-called multidetector CT (MDCT) refers to the newest generation of computed tomographs, having been available in clinical radiology since 1998. Multidetector CT is widely available and distinguished by a high, virtually isotropic resolution (pixel size 0.5-1 mm in each spatial direction), and this makes it possible to view the CT images in any spatial plane. The examination time varies between 1 and 10 seconds, giving rise to images virtually free of artefacts, even when there is dyspnoea or lack of patient cooperation. The latest MDCT scanners are equipped with “dual-energy” technology, in which two different energies/tube voltages are used simultaneously. Owing to the energy dependence of absorption, it is possible to highlight certain tissue properties, for example the distribution of iodine after administration of contrast agent as surrogate for regional blood circulation.

According to some embodiments, a criterion in the present invention is the automation. As already set out, CTEPH is a rare condition which is underdiagnosed. Non-detection of this condition can have fatal consequences for the patient. Therefore, according to some embodiments of the present invention, computed tomography images of the thorax are analysed in an automated manner for signs of the presence of CTEPH. What is meant here by “automated” is that no human intervention at all is required. According to some embodiments of the invention, a computer program is thus installed on a computer system that has access to computed tomography images of the thorax, runs as a background process, and analyses the images in an automated manner for signs of the presence of CTEPH. A background process refers to a process that does not act directly with the user and hence acts asynchronously to the user interface.

In a first step of the method according to some embodiments of the invention, one or more computed tomography images of the thorax of a person are received or retrieved. Customarily, a CT image is a data set, by means of which the structures of the thorax of the person can be depicted three-dimensionally. Thus, a CT image customarily represents the thorax of the person at the time of recording of the computed tomogram. Multiple CT images can represent the pulmonary region of the person at different times; on the basis of these multiple CT images, it is possible therefore to identify temporal changes in the tissue structures and thus, for example, to examine a progression of a disease. However, it is also conceivable that the multiple CT images are CT images representing different regions of the thorax.

As already described, computed tomography is a customary method for the diagnosis of heart and lung diseases. It is therefore conceivable to examine CT images already present in a database for the presence of signs of CTEPH. In some embodiments of the present invention, CT images present in one or more databases are retrieved and are analysed for the presence of signs of CTEPH. For example, this can occur at regular intervals. It is, for example, conceivable to carry out at regular intervals, for example every day or every week, a search for new CT images in the databases in which CT images are customarily deposited and to retrieve the new CT images for an image analysis. However, the retrieval can also take place irregularly. The retrieval can also be triggered by an event, for example by the storing of a new CT image. Preferably, the retrieval of new CT images is done in an automated manner

It is also conceivable to carry out the analysis according to some embodiments of the invention for the presence of signs of CTEPH as a kind of standard analysis for each CT image generated from the thorax of a person. According to some embodiments of the present invention, a CT image generated from the thorax of a person is, after its generation, immediately and automatically subjected to an image analysis according to some embodiments of the invention. In such a case, a computer system oriented to generating a relevant CT image can be configured such that it forwards the CT image to the image analysis according to some embodiments of the invention. The components executing the image analysis receive the CT image.

In a further step of the method according to some embodiments of the invention, an automated analysis of the CT image takes place. The analysis is carried out by an image recognition software. The image recognition software is configured such that it examines the CT image for the presence of specific (characteristic) features.

CT images of persons suffering from CTEPH often show characteristic features that are not exhibited by persons not suffering from CTEPH. According to some embodiments of the invention, the CT images are examined for the presence of said characteristic features.

A characteristic feature that can be identified in the analyses mentioned is the ratio of the volumes and/or the diameters of right ventricle and left ventricle (RV/LV ratio) (see, for example, Gonzales G et al. PLoS ONE 10(5): e0127797). A value of 0.9 or more in the RV/LV diameter ratio is an indication of the presence of CTEPH. A further characteristic feature is the degree of curvature of the interventricular septum (see, for example, D. A. Moses et al., Quantification of the curvature and shape of the interventricular septum; Magnetic Resonance in Medicine, Vol. 52 (1), 2004, 154-163 and F. Haddad et al.: Septal Curvature is a marker of hemodynamic, anatomical, and electromechanical ventricular interdependence in patients with pulmonary arterial hypertension, Echocardiography Vol. 31(6) 2014, 699-707). It is also possible to determine the ratio of the diameters of the pulmonary artery and the aorta (PA:A ratio) at the point at which the pulmonary artery branches off (see, for example, A. S. Iyer et al.: CT scan-measured pulmonary artery to aorta ratio and echocardiography for detecting pulmonary hypertension in severe COPD, CHEST 2014, Vol. 145(4), 824-832). A PA:A ratio of 0.7 or greater is a further indication of the presence of CTEPH. Characteristic vascular features are, for example, the lack of contrast agent in the distal vessel sections in the case of total obstruction or the formation of rope-ladder thrombi, meshes, stenoses and partial obstructions. The CTEPH-specific parenchymal features include scars, mosaic perfusion, ground glass opacity and bronchial anomalies. The scars arise as a result of infarctions owing to the occlusion of pulmonary vessels that are usually localized in the lower segments. Mosaic perfusion consists of regions of different density resulting from regions of irregular hypo- and hyperperfused regions caused by embolic occlusions, vascular rearrangement of the distal vessels and compensation mechanisms. Hypoperfused regions are observed particularly in the distal direction from the occluded vessels since blood flow and hence the concentration of the contrast agent are reduced in these regions. Hyperdense areas are usually apparent in regions that are now hyperperfused owing to the redistribution and stand out as ground glass opacity. The latter and further characteristic features are described in the literature (see, for example, J. E. Leifheit, Characterization of ground glass opacity in patients with chronic thromboembolic pulmonary hypertension compared to pulmonary hypertension of other WHO classification using Dual Energy CT [in German], Inaugural Dissertation for Attaining the Degree of Doctor of Medicine at the Justus Liebig University of Giessen, 2017).

The characteristic features are preferably identified by conventional pattern recognition methods. In principle, machine learning methods are also conceivable (artificial neural networks, deep learning and the like). The number of available CT images of persons suffering from CTEPH is, however, (still) comparatively low, meaning that possibly the low number of available data for training could cause problems for the machine learning methods. In principle, it is, however, conceivable to first identify the CTEPH-characteristic features in the CT images using pattern recognition methods, to subject persons whose CT images exhibit CTEPH-characteristic features to a further diagnostic procedure, and to forward the CT images from those persons for whom the further diagnostic procedure confirmed the presence of CTEPH to a training set for machine learning in order to constantly lower the error rates for the machine learning-based image recognition software. If the error rates for the machine learning-based image recognition software are lower than for the image recognition software based on pattern recognition, a switch can be made to the machine learning-based image recognition software.

It is conceivable that, in the image analysis, no CTEPH-specific features are found in the CT images examined. In such a case, it is possible to store in a database, in relation to the relevant CT image or in relation to the person from whom the CT image was generated, an item of information indicating that no CTEPH-specific features were identified in the CT image.

If features indicating the presence of CTEPH were found, it is possible to store in a database, in relation to the relevant CT image or in relation to the person from whom the CT image was generated, an item of information indicating that CTEPH-specific features were identified in the CT image.

In a further step of the method according to some embodiments of the invention, a calculation of a probability of the presence of CTEPH is effected on the basis of the characteristic features ascertained. A value of 100% indicates that the patient is suffering from CTEPH; a value of 0% indicates that CTEPH can be ruled out.

The probability can be calculated by following many different approaches. It is conceivable, for example, that the one or more CT images are examined for the presence of a number of characteristic features. The probability can be calculated from the sum of the number of features found divided by the sum of the number of features tested (P=(sum of features found)/(sum of features tested)). In such an approach, all features are of equal value. Alternatively, it is conceivable that the individual features are weighted with a factor, such that features that are more likely to indicate CTEPH are given a higher value in the probability function than features that are less specific. It is also conceivable that the severity of a feature is determined; where the severity indicates the extent in respect of the presence of CTEPH. The higher the degree (the extent), the clearer the manifestation of the feature and the higher the probability of the presence of CTEPH. It is also conceivable that one or more decision trees or regression trees are run; it may be the case that one feature indicates CTEPH only in combination with another feature. Further methods and combinations of methods of ascertaining the probability are conceivable.

If the probability is above a defined threshold, a message is generated that gives information that the person who was the subject of the CT images has a defined probability of suffering from CTEPH and therefore further examinations should be undertaken to clarify the finding. The threshold value may, for example, be between 20% and 70%. It is preferably above 20% and below 51%.

According to some embodiments of the invention, a message that the person should be subjected to a further diagnostic procedure in order to confirm CTEPH or to reliably rule it out is then communicated. Said message can, for example, be addressed to the person from whom the relevant CT image originates. However, it can also be addressed to his/her physician or hospital carer or to another person who is in contact with the person for whom there are signs of CTEPH. The communication of the message can be a text message (e-mail, SMS, etc.) or voice message.

### BRIEF DESCRIPTION OF THE DRAWINGS

The invention is described in detail hereinafter with reference to figures and examples, without wishing to restrict the invention to the features or combinations of features described and shown.

FIG. 1 shows an implementation of the system according to some embodiments of the invention.

FIG. 2 shows a schematic view of a system according to some embodiments of some embodiments of the invention.

FIG. 3 shows a schematic view of a computer system according to some embodiments of the invention.

### DETAILED DESCRIPTION OF EXEMPLARY EMBODIMENTS

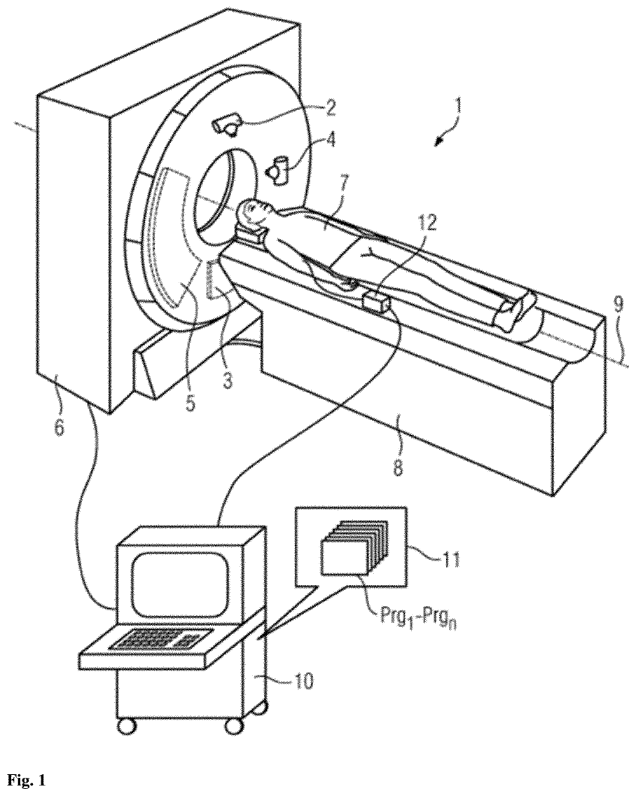

FIG. 1 shows an implementation of the system according to some embodiments of the invention.

FIG. 1 shows a CT system 1 executed as a twin-focus detector system. It has a first x-ray tube 2 with an opposite detector and a second x-ray tube 4 with a further opposite detector 5. Both focus/detector systems 2, 3 and 4, 5 are arranged in a gantry housing 6 on a gantry which rotates about a system axis 9 and is not visualized here. The patient 7 is on a longitudinally movable patient bed 8. Before the scanning of the patient 7, to improve the contrast of a CT image reconstructed from the detector output data, a contrast agent is administered to the patient 7 by means of a contrast agent injector 12. The control of the overall CT system and if necessary also the evaluation of the detector data and the reconstruction of the CT image as section images or volume data can be effected by a control and computing unit 10. This control and computing unit 10 has a memory 11 which stores, as well as the detector output data measured, computer program data Prg1-Prgn that are executed in operation and essentially assume the function of controlling the system and the evaluation of the data.

In some embodiments of the present invention, the computer program runs as a background process on the control and computing unit 10. It analyses the section images or volume data for the presence of CTEPH indications. If CTEPH indications are identified, and a calculated probability of the presence of CTEPH is above a defined threshold, the computer program according to some embodiments of the invention creates a message on the screen of the control and computing unit 10 that informs the radiologist that there is a suspicion of CTEPH.

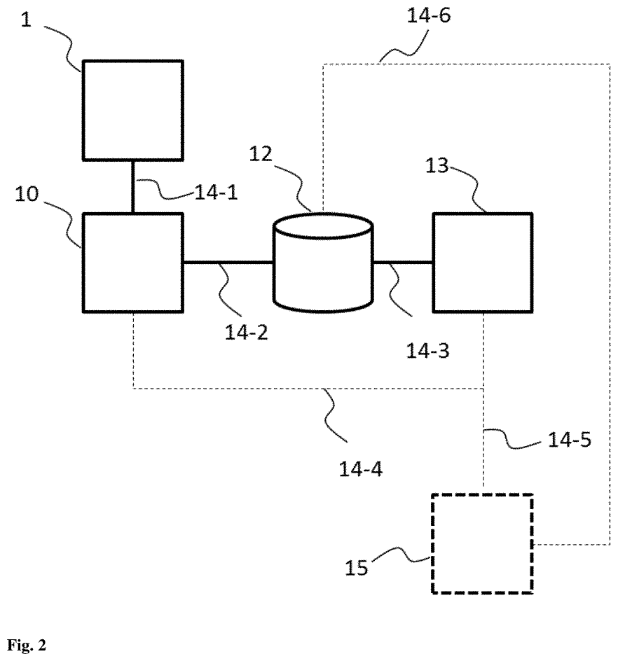

FIG. 2 shows a schematic view of a system according to some embodiments of the invention.

The CT system 1 is connected via the connection 14-1 to the control and computing unit 10. The control and computing unit 10 controls the CT system 1 and evaluates the detector data and reconstructs the CT image as section images or volume data. The section images and volume data are stored in a database 12 to which the control and computing unit 10 is connected via the connection 14-2. It is also conceivable that the database is part of the control and computing unit 10. The computer system 13 can also access the database 12 via the connection 14-3. The computer program according to some embodiments of the invention is running on the computer system 13. It is configured such that it analyses the CT images of a human thorax stored in the database 12 for indications of the presence of CTEPH. If no indications are identified, corresponding information relating to the CT images is stored. If indications of the presence of CTEPH are identified, the corresponding information relating to the CT images is likewise stored.

The computer program installed and running on the computer system 13 is configured such that, on the basis of the features ascertained that indicate CTEPH, a probability of the presence of CTEPH is calculated. If this probability is above a defined threshold, the computer program creates a message that CTEPH could be present.

The computer program installed and running on the computer system 13 may be configured such that it displays the communication as to the presence of CTEPH indications on a screen which is part of the computer system 13. It is also conceivable that the computer program is configured such that it transmits a message of the presence of CTEPH indications via the connection 14-4 to the control and computing unit 10, via which the message is then displayed, for example on a screen. It is also conceivable that the computer system 10 draws the information as to whether CTEPH indications are present directly from the database 12. It is also conceivable that the computer program is configured such that it transmits a message of the presence of CTEPH indications via the connection 14-5 to a further computer system 15, via which the message is then displayed, for example via a screen. It is also conceivable that the computer system 15 draws the information as to whether CTEPH indications are present from the database 12 via the connection 14-6. The dotted components in FIG. 2 are optional. The connections 14-1, 14-2, 14-3, 14-4, 14-5 and 14-6 may be cable connections, glass fibre-based connections and/or wireless connections (e.g. via radio).

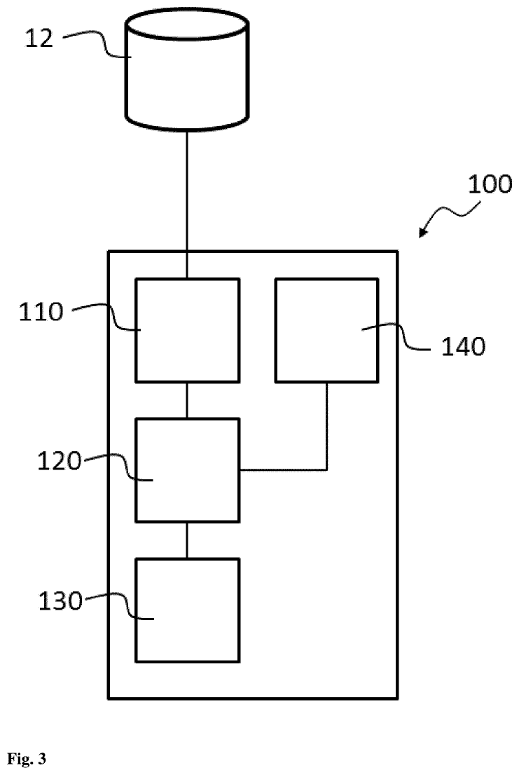

FIG. 3 shows a schematic view of a computer system according to some embodiments of the invention.

The computer system 100 is connected to a database 12 on which the computed tomography images of a human thorax are stored. It is also conceivable that the database 12 is part of the computer system 100. The computer system 100 includes a receiver unit 110 with which the computed tomography images can be received or retrieved. The computer system 100 includes a control and computing unit 120 by which the computed tomography images can be analysed and by which features in the computed tomography images that indicate the presence of CTEPH can be recognized. The computer system 100 includes a computing and checking unit 130 by which a probability of the presence of CTEPH can be calculated and by which it can be checked whether the probability is above a defined threshold. The computing and checking unit 130 may be part of the control and computing unit 120. The computer system 100 includes an output unit 140 by which a communication as to the result of the analysis can be displayed to a person or transmitted to a person.

Further embodiments of the present invention are:

1. Method for identifying signs of the presence of CTEPH in a person, comprising the following steps:
receiving or retrieving one or more computed tomography images of the thorax of the person
automatically analysing the one or more computed tomography images by means of an image recognition software
determining features in the one or more computed tomography images, which features indicate the presence of CTEPH
communicating a message to the person and/or another person for further clarification of the finding.
   2. Method according to Embodiment 1, wherein computed tomography images present in databases are retrieved and are forwarded to automatic analysis.
   3. Method according to Embodiment 1, wherein computed tomography images are, after their generation, received from that computer system which generated the computed tomography images and are forwarded to automatic analysis.
   4. Computer system for identifying signs of the presence of CTEPH in a person, comprising:
means for the receipt or retrieval of one or more computed tomography images of the thorax of the person
means for the automated analysis of the one or more computed tomography images
means for the automated identification of features in the one or more computed tomography images, which features indicate the presence of CTEPH
means for the communication of a message to the person and/or another person for further clarification of the finding.
   5. Computer program product comprising a data carrier on which there is stored a computer program which can be loaded into the memory of a computer system, where it causes the computer system to execute the following steps:
receiving or retrieving one or more computed tomography images of the thorax of a person
automatically analysing the one or more computed tomography images by means of an image recognition software
determining features in the one or more computed tomography images, which features indicate the presence of CTEPH
communicating a message to the person and/or another person for further clarification of the finding.

1. A method for identifying signs of the presence of chronic thromboembolic pulmonary hypertension (CTEPH) in a person, comprising:
receiving or retrieving one or more computed tomography images of the thorax of the person,
analysing the one or more computed tomography images via image recognition software,
determining one or more features in the one or more computed tomography images, the one or more features indicating presence of CTEPH,
calculating a probability of the presence of CTEPH based on the one or more determined features, and
communicating a message to at least one of the person and another person for further assessment if the probability is above a defined threshold,
wherein the method is performed automatically as one or more background processes in one or more computer systems.

2. The method of claim 1, wherein the one or more computed tomography images are automatically retrieved from one or more databases.

3. The method of claim 1, wherein the one or more computed tomography images are received from a computer system that generated the one or more computed tomography images.

4. The method of claim 1, wherein at least one characteristic feature is identified via pattern recognition.

5. The method of claim 4, wherein the at least one characteristic feature is selected from the following list: ratio of volumes of a right ventricle and a left ventricle, ratio of diameters of the right ventricle and the left ventricle, a degree of curvature of an interventricular septum, ratio of diameters of a pulmonary artery and an aorta at a level at which the pulmonary artery branches off, presence of stenosis, severity of stenoses, presence of mosaic perfusion, severity of mosaic perfusion, presence of ground glass opacity, and severity of ground glass opacity.

6. The method of claim 1, wherein the one or more features are determined via machine learning.

7. A computer system for identifying signs of the presence of chronic thromboembolic pulmonary hypertension (CTEPH) in a person, the computer system configured for:
automatically receiving or retrieving one or more computed tomography images of the thorax of the person,
automatically analyzing the one or more computed tomography images,
automatically identifying features in the one or more computed tomography images, identified features indicating presence of CTEPH,
automatically calculating a probability of the presence of CTEPH based on the identified features, and
automatically communicating a message to at least one of the person and another person for further assessment.

8. The computer system of claim 7, comprising:
a receiver for receiving or retrieving the one or more computed tomography images,
a control and computing unit for analyzing the one or more computed tomography images and identifying features in the one or more computed tomography images that indicate the presence of CTEPH,
a computing and checking unit for calculating a probability of the presence of CTEPH and checking whether the probability is above a defined threshold,
an output unit for displaying or transmitting a result of the analysis to at least one of the person or another person.

9. The computer system of claim 7, wherein the computer system is configured for receiving or retrieving the one or more computed tomography images, analyzing the one or more computed tomography images, identifying features in the one or more computed tomography images, calculating the probability of the presence of CTEPH, and communicating the message to at least one of the person and another person for further assessment as one or more background processes without human intervention.

10. The computer system according to of claim 7, wherein the computer system comprises a computed tomography system.

11. The computer system of claim 10, wherein the computer system is connected to a database that stores the one or more computer tomography images generated from the computer tomography system.

12. A non-transitory computer readable medium storing a computer program that loads into the memory of a computer system, wherein the computer program includes instructions for:
receiving or retrieving one or more computed tomography images of the thorax of the person,
analysing the one or more computed tomography images via image recognition software,
determining features in the one or more computed tomography images, the features indicating presence of CTEPH,
calculating a probability of the presence of CTEPH based on the determined features, and
communicating a message to at least one of the person and another person for further assessment if the probability is above a defined threshold,
wherein the instructions of the computer program are configured to operate automatically as one or more background processes on the computer system.
